(19) United States
(12) Patent Application Publication
(10) Pub. No.: US 2012/0289740 A1
(43) Pub. Date: Nov. 15, 2012

(54) Catalyst, Method for Manufacturing the Same by Supercritical Fluid and Method for Hydrogenating an Aromatic Compound by Using the Same

(75) Inventors: Chung-Sung Tan, Hsinchu City (TW); Yu-Wen Chen, Jhongli City (TW); Hsin-Wei Lin, Hsinchu City (TW); Clive Hsu Yen, Hsinchu City (TW)

(73) Assignee: NATIONAL CENTRAL UNIVERSITY, Jhongli City (TW)

(21) Appl. No.: 13/405,613

(22) Filed: Feb. 27, 2012

(30) Foreign Application Priority Data
May 13, 2011 (TW) 100117028

(51) Int. Cl. B01J 23/46; B01J 21/08; C07C 51/36; C07C 5/11; B01J 23/44; C07C 29/20; B82Y 40/00; B82Y 30/00

(52) U.S. Cl. 562/400; 502/325; 502/339; 502/261; 502/262; 568/816; 585/269; 977/773; 977/810; 977/902; 977/896

(57) ABSTRACT

Disclosed herein is a method for manufacturing a catalyst. The catalyst includes a mesoporous support and a plurality of metal nanoparticles dispersed and positioned in the mesopores of the mesoporous support. The method comprises the steps of: (a1)) allowing an organometallic precursor to be in contact with a mesoporous support, in which the organometallic precursor includes at least one material selected from the group consisting of ruthenium-containing compound, rhodium-containing compound and palladium-containing compound; and (a2) reducing the organometallic precursor in the presence of a supercritical fluid with a reductant, so that the organometallic precursor is reduced to the metal nanoparticles.

### CROSS-REFERENCE TO RELATED APPLICATION

This application claims the priority benefit of Taiwan application serial no. 100117028, filed May 13, 2011, the full disclosure of which is incorporated herein by reference.

### BACKGROUND

1. Technical Field

The present disclosure relates to a method for manufacturing a catalyst and a method for hydrogenating an aromatic compound. More particularly, the present disclosure relates to a method for manufacturing a catalyst by supercritical fluid and a method for hydrogenating an aromatic compound by using the catalyst.

2. Description of Related Art

Methods of using supercritical fluids (SCF) for synthesizing nanomaterials have been developed rapidly in recent years. Several review articles summarized numerous supercritical fluid techniques for preparation of different nanomaterials. Of all the SCF techniques available, chemical fluid deposition (CFD), also known as supercritical fluid deposition (SCFD), has probably the highest opportunity for mass production due to its simplicity. The CFD technique was developed by Watkins et al. in 1995 for making metal nanoparticles or films by using supercritical carbon dioxide (J. J. Watkins and T. J. McCarthy, Chem. Mater. 7, 1991 (1995)). The advantage of using supercritical carbon dioxide (scCO₂) as a “green” medium is also important since it is non-toxic, recyclable, and creates minimum liquid waste.

Ordered mesoporous silica materials have drawn much attention in the nanotechnology field nowadays. Their pore diameters are in the range of 2 to 50 nm so they are suitable supports for the immobilization of the nanoparticles. MCM-41 (Mobil Composition of Matter) is an ordered mesoporous silica material discovered by researchers of Mobil in 1992. The material contains linear mesoporous channels packed as hexagonal arrays. However, synthesizing nanoparticles inside these small pores may be challenging. When metal precursors are dissolved in solvents, they have limiting diffusion to penetrate inside the silica pores.

According to a recent review article, which summarized many methods of growing different metal nanoparticles in mesoporous silica materials, using SCF methods seems to be very promising of making homogeneous nanocomposite materials (Q. Wang and D. F. Shantz, J. Solid State Chem. 181, 1659 (2008)). Some previous researches have demonstrated the advantages of using SCF for making the nanocomposites. Chatterjee et al. synthesized gold nanoparticles in the pores of MCM-48 by using CFD method (M. Chatterjee, Y. Ikushima, Y. Hakuta and H. Kawanami, Adv. Synth. Catal. 348, 1580 (2006)). In their work, they showed that the density of supercritical CO₂ is an important factor related to the average gold particle size. Dhepe et al. fabricated rhodium and rhodium-platinum alloy particles in HMM-1 and FSM-16 (P. L. Dhepe, A. Fukuoka and M. Ichikawa, Phys. Chem. Chem. Phys. 5, 5565 (2003)). They showed that using a supercritical fluid treatment after impregnation can obtain high dispersion of the nanoparticles inside the mesoporous channels. When the SCF treatment was neglected, the nanoparticles mainly grew on the outer surfaces of the silica. Wakayama and Fukushima prepared nanoparticles of Pd, Rh, Ru and Fe₂O₃ in nanoporous silica, FSM-16 (H. Wakayama and Y. Fukushima, J. Chem. Eng. Jpn. 42, 134 (2009)). Their experiment results showed that the nanoparticles all have average sizes slightly smaller than the pore sizes of FSM-16. This could be suggested that the nanoparticles were located inside the pores of FSM-16. The applications of the nanocomposites can be very broad and one of the common applications is to use them as chemical reaction catalysts, such as in hydrogenation reactions. Ring hydrogenation of aromatic compounds has been an important part in the chemical industry. Many pollutants, which contain aromatic rings, can be treated by hydrogenation in order to transform them into saturated compounds which have less toxicity and can be decomposed more easily in the environment. Niederer et al. reported MCM-41 supported Pd, Ir and Rh nanoparticles for the hydrogenation of cyclic olefins (J. P. M. Niederer et al., Top. Catal. 18, 265 (2002)). Others also had demonstrated mesoporous aluminosilicates (Al-MCM-41) supported metal nanoparticles for the hydrogenation of phenol and naphthalene (M. Chatterjee et al., Adv. Synth. Catal. 351, 1912 (2009); and S. Albertazzi et al., J. Mol. Catal. A-Chem. 200, 261 (2003)).

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

According to a first aspect of the present disclosure, a method for manufacturing a catalyst is disclosed. The catalyst includes a mesoporous support and a plurality of metal nanoparticles dispersed and positioned in the mesopores of the mesoporous support. The method comprises the steps of:

(a1) allowing an organometallic precursor to be in contact with a mesoporous support, in which the organometallic precursor comprises at least one material selected from the group consisting of ruthenium-containing compound, rhodium-containing compound and palladium-containing compound; and

(a2) reducing the organometallic precursor in the presence of a supercritical fluid with a reductant, so that the organometallic precursor is reduced to the metal nanoparticles.

According to a second aspect of the present disclosure, a catalyst is provided. The catalyst is produced by any embodiment or example described hereinbefore, and is capable of hydrogenating an organic compound.

According to a third aspect of the present disclosure, a method for hydrogenating an aromatic compound is disclosed. In one embodiment, the method comprises the steps of:

(b1) providing a catalyst that is manufactured by any embodiment or example described hereinbefore; and

(b2) hydrogenating the aromatic compound with hydrogen in the presence of the catalyst and a solvent.

In another embodiment, the method of hydrogenating an aromatic compound comprises the steps of:

(c1) providing a catalyst having a plurality of metal nanoparticles dispersed therein, the catalyst being manufactured by a method comprising the steps of allowing an organometallic precursor to be in contact with a mesoporous support, and reducing the organometallic precursor in the presence of a supercritical fluid with a reductant so that the organometallic precursor is reduced to the metal nanoparticles; and

(c2) hydrogenating the aromatic compound with hydrogen in the presence of the catalyst and water in liquid phase, wherein the catalyst is dispersed in the water.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIGS. 1A-1F are TEM images respectively illustrating the catalysts of EXAMPLES 1-6;

FIG. 2 and FIG. 3 respectively illustrates patterns of powder and small angle X-ray diffraction associated with EXAMPLES 1-6; and

FIG. 4a and FIG. 4b respectively illustrate the BPA conversion and the HBPA yield in EXAMPLE 21 and COMPARATIVE EXAMPLE 3.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

According to a first aspect of the present disclosure, a method for manufacturing a catalyst is disclosed. The catalyst includes a mesoporous support and a plurality of metal nanoparticles dispersed and positioned in the mesopores of the mesoporous support. The method comprises the steps of:

(a1) allowing an organometallic precursor to be in contact with a mesoporous support, in which the organometallic precursor comprises at least one material selected from the group consisting of ruthenium-containing compound, rhodium-containing compound and palladium-containing compound; and

(a2) reducing the organometallic precursor in the presence of a supercritical fluid with a reductant, so that the organometallic precursor is reduced to the metal nanoparticles.

In the step (a1), the mesoporous support is a particular substance having a plurality of mesopores. The term “mesopores” herein refers to pores having an averaged diameter of greater than 2 nm and less than 50 nm. In one embodiment, the mesoporous support may be made of silica, and comprise a hexagonal array of mesopores. The diameter of each of the mesopores may be ranged from about 2 nm to about 4 nm. Specifically, MCM-41 (trade name) provided by SIGMA-ALDRICH Co. LLC may be used.

The organometallic precursor comprises at least one of ruthenium-containing compound, rhodium-containing compound, and palladium-containing compound. Suitable materials for the organometallic precursor include, but are not limited to, ruthenium acetylacetonate (hereinafter also refers to “Ru(acac)₃”), rhodium acetylacetonate (hereinafter also refers to “Rh(acac)₃”), palladium acetylacetonate (hereinafter also refers to “Pd(acac)₂”), bis(2,2,6,6-tetramethyl-3,5-heptanedionato)(1,5-cycloctadiene)ruthenium (hereinafter also refers to “Ru(cod)(tmhd)₂”), acetylacetonato(1,5-cyclooctadiene) rhodium (hereinafter also refers to “Rh(cod)(acac)”), palladium hexafluoroacetylacetonate(hereinafter also refers to “Pd(hfac)₂”) and any combination thereof.

The approach of allowing the organometallic precursor to be in contact with the mesoporous support is non-limited. For instance, a mixing process or an adsorption process may be employed to make the organometallic precursor touches the mesoporous support. The mixing process and the adsorption process may be performed at atmosphere or at a pressure higher than or less than an ambient pressure. In one embodiment, the step (a1) may involve simply mixing the organometallic precursor and the mesoporous support at atmosphere such that the organometallic precursor is in contact with the mesoporous support. In another embodiment, the step (a1) may comprise the steps of: (i) mixing the organometallic precursor and the mesoporous support with a solvent at atmosphere, so that the organometallic precursor is dissolved and/or dispersed in the solvent; and then (ii) removing the solvent such that the organometallic precursor is adsorbed and/or attached onto the mesoporous support. In particular, stirring or ultrasonicating treatment may be employed in the step (i) to mix the organometallic precursor and the mesoporous support in the solvent. Furthermore, vacuum dry may be used to remove the solvent in the step (ii).

In the case where the organometallic precursor is any of Ru(cod)(tmhd)₂, Rh(cod)(acac), Pd(hfac)₂ and a combination thereof, the step (a1) may involve simply adding the organometallic precursor to the mesoporous support since each of Ru(cod)(tmhd)₂, Rh(cod)(acac) and Pd(hfac)₂ exhibits a high solubility in supercritical fluid.

In the step (a2), the organometallic precursor is reduced to metal nanoparticles by a reductant in the presence of a supercritical fluid. The term “supercritical fluid” herein refers to a gas above its critical pressure and above its critical temperature. In one embodiment, the step (a2) involves exposing the organometallic precursor and the mesoporous support to a mixture of the supercritical fluid and the reductant. In another embodiment, the step (a2) comprises the following steps in sequence: subjecting the organometallic precursor and the mesoporous support in a container; introducing the supercritical fluid into the container; and introducing the reductant into the container having the supercritical fluid therein.

It is necessary that the reduction of the organometallic precursor occur in the presence of the supercritical fluid. The catalyst activity may be dramatically and unexpectedly increased because of the presence of the supercritical fluid during the reduction of the organometallic precursor. Without being bound to any theory, it is believed that the nanoparticles is redispersed in the mesoporous support by the supercritical fluid during the reduction of the organometallic precursor, and results in a more uniform distribution of metal nanoparticles so that the catalyst activity is dramatically increased.

Furthermore, the supercritical fluid and the reductant form a mixture that facilitates controlling the formation of the metal nanoparticles in the mesopores of the mesoporous support. In examples, a molar ratio of the reductant to the supercritical fluid is about 0.1:1 to 1:1. When the molar ratio of the reductant to the supercritical fluid exceeds this range, the particle size of the metal nanoparticle may be too large and is unfavorable to the activity of the catalyst. The particle size of the metal nanoparticle may have a wide distribution in this case. On the other hand, when the molar ratio of the reductant to the supercritical fluid is below this range, the reaction rate of the reduction reaction may be too slow.

In one embodiment, the reductant and the supercritical fluid are respectively hydrogen gas and supercritical carbon dioxide. The reduction reaction of the organometallic precursor may be performed at a total pressure of about 100-400 bar, in which the partial pressures of hydrogen and carbon dioxide are respectively about 20-150 bar and 100-250 bar, at a temperature of about 100° C. to about 300° C., specifically about 150-250° C. The reduction reaction may be carried out for a time period of about 1-3 hours.

The step (a2) may be performed in a batch reactor, semi-batch reactor or continuous reactor. In one embodiment, the reduction reaction of the organometallic precursor is performed in a batch reactor, and a molar ratio of the reductant to the organometallic precursor is greater than or equal to about 10:1 for increasing the reaction rate of reduction reaction.

In some embodiments, the metal nanoparticles of the catalyst exists in a concentration of 0.5-10% by weight of the catalyst, specifically about 3-6% by weight of the catalyst. The diameter of the metal nanoparticles is less than about 10 nm, specifically about 2-8 nm.

According to a second aspect of the present disclosure, a catalyst is provided. The catalyst is produced by any embodiment or example described hereinbefore, and is capable of hydrogenating an organic compound. The catalyst is characterized in providing a turnover number of greater than 600 and a turnover frequency of greater than 200 hr⁻¹. The term “turnover number” herein is defined as the molar numbers of the consumed organic compound divided by the molar numbers of the metal nanoparticles in the catalyst. The term “turnover frequency” herein is the turnover number divided by the reaction time period in hours. In some embodiments, the catalyst may provide a turnover number of greater than 2000 and a turnover frequency of greater than 1400 hr⁻¹.

According to a third aspect of the present disclosure, a method for hydrogenating an aromatic compound is disclosed. In one embodiment, the method comprises the steps of:

(b1) providing a catalyst that is manufactured by any embodiment or example described hereinbefore; and

(b2) hydrogenating the aromatic compound with hydrogen in the presence of the catalyst and a solvent.

In another embodiment, the method of hydrogenating an aromatic compound comprises the steps of:

(c1) providing a catalyst having a plurality of metal nanoparticles dispersed therein, the catalyst being manufactured by a method comprising the steps of allowing an organometallic precursor to be in contact with a mesoporous support, and reducing the organometallic precursor in the presence of a supercritical fluid with a reductant so that the organometallic precursor is reduced to the metal nanoparticles; and

(c2) hydrogenating the aromatic compound with hydrogen in the presence of the catalyst and water in liquid phase, wherein the catalyst is dispersed in the water.

Exemplary aromatic compounds which may be hydrogenated by the method described above include but are not limited to 4,4′-isopropylidenediphenol (BPA), 4,4′-methylenediphenol (BPF), benzoic acid and p-xylene.

In one embodiment, the mesoporous support of the step (c1) may be made of a material that is capable of forming hydrogen bonding with water. The water may cooperate with the mesoporous support to create a suitable chemical environment for the hydrogenation of the aromatic compound. Without being bound to any theory, it is believed that the hydrogen bonds may facilitate the water molecules surrounding the catalyst, and thus creating a better dispersion of the catalyst. In one example, the conversion of the aromatic compound is nearly 100% in a reaction time period of 4 hours, and the turnover frequency associated with hydrogen may be over 340 hr⁻¹. The term “turnover frequency associated with hydrogen” herein refers to the molar numbers of hydrogen gas consumed in the hydrogenation reaction in one hour divided by the molar numbers of the metal nanoparticles in the catalyst. In one example, the mesoporous support may be made of silica, and comprise a hexagonal array of mesopores. Specifically, MCM-41 described hereinbefore may be used.

In examples, the organometallic precursor of the step (c1) may comprise any of Ru(acac)₃, Rh(acac)₃, Ru(cod)(tmhd)₂, Rh(cod)(acac) and a combination thereof. The metal nanoparticles of the catalyst may comprise one of ruthenium, rhodium and ruthenium-rhodium alloy.

In one embodiment, the step (c2) comprises the steps of allowing the aromatic compound and the catalyst to be in contact with the water to form a mixture, and introducing the hydrogen into the mixture.

### EXAMPLES

The following Examples are provided to illustrate certain aspects of the present invention and to aid those of skill in the art in practicing this invention. These Examples are in no way to be considered to limit the scope of the invention in any manner.

Journal of Nanoscience and Nanotechnology, Vol. 11, 2465-2469, 2011, Chung-Sung Tan et al. and Catalyst Today, Vol. 174, 121-126, 2011, Chung-Sung Tan et al., both of which are incorporated herein by reference.

### Preparation and Characterization of the Catalysts

### Examples 1-6

Metal acetylacetonates were used as the CO₂-soluble organometallic precursors in EXAMPLES 1-6. In EXAMPLES 1-3, the organometallic precursors were respectively Ru(acac)₃, Rh(acac)₃ and Pd(acac)₂. In EXAMPLES 4-6, the organometallic precursors were respectively a mixture of Ru(acac)₃ and Rh(acac)₃, a mixture of Ru(acac)₃ and Pd(acac)₂, and a mixture of Rh(acac)₃ and Pd(acac)₂, as listed in Table 1. In each example of Examples 1-6, MCM-41 and the corresponding organometallic precursor(s) (total weight 300 mg) were added into a rounded bottom flask to form a mixture. The content of the metal element of the corresponding organometallic precursors in the each mixture was 5 wt. % in total. In EXAMPLES 4-6, the two metal elements of the two organometallic precursors were of equal weight. Tetrahydrofuran (THF) was then added into the flask and followed by an ultrasonicating process to disperse and dissolve the organometallic precursors. Afterwards, THF was removed by using a rotary vacuum evaporator. The remaining sample powder was then transferred into a high-pressure stainless steel cell located in an oven. At a temperature of 200° C., a mixture of hydrogen gas and carbon dioxide, in which the partial pressures of hydrogen and carbon dioxide are respectively 500 psi and 2500 psi, were injected into the cell for a reaction time of 2 hours so that the organometallic precursors were reduced to metal nanoparticles. After the reaction, the cell was depressurized and flushed with CO₂ for a few times to eliminate the unreacted organometallic precursors. Monometallic (Ru, Rh, Pd) nanoparticles were obtained in EXAMPLES 1-3, whereas bimetallic (Ru—Rh, Ru—Pd, Rh—Pd) nanoparticles were obtained in EXAMPLES 4-6.

TABLE 1

| | | | Averaged |
|---|---|---|---|
| | | | particle |
| | | | size of metal |
| | Organometallic | | nanoparticles |
| | precursor | Catalystst | (nm) |
| EXAMPLE 1 | Ru(acac)₃ | Ru/MCM-41 | 2.7 ± 0.4 |
| EXAMPLE 2 | Rh(acac)₃ | Rh/MCM-41 | 7.8 ± 3.5 |
| EXAMPLE 3 | Pd(acac)₂ | Pd/MCM-41 | 3.7 ± 1.1 |
| EXAMPLE 4 | Ru(acac)₃ + Rh(acac)₃ | Ru-Rh/MCM-41 | 3.4 ± 1.2 |
| EXAMPLE 5 | Ru(acac)₃ + Pd(acac)₂ | Ru-Pd/MCM-41 | 2.9 ± 0.6 |
| EXAMPLE 6 | Rh(acac)₃ + Pd(acac)₂ | Rh-Pd/MCM-41 | 3.8 ± 0.6 |

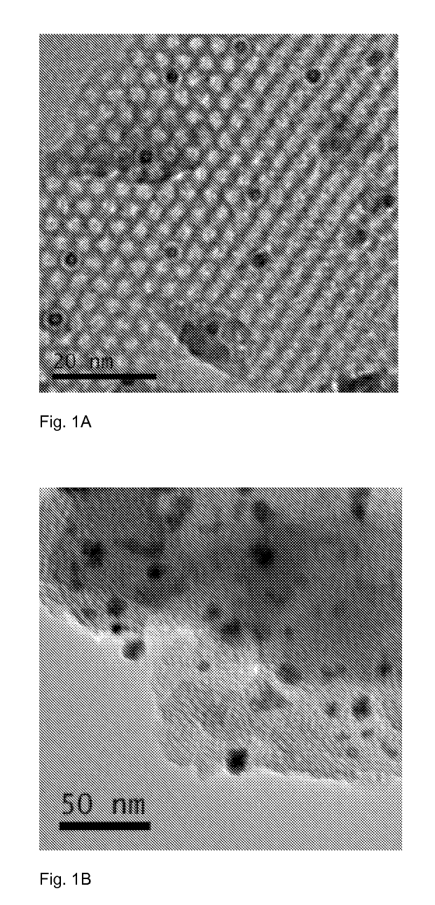

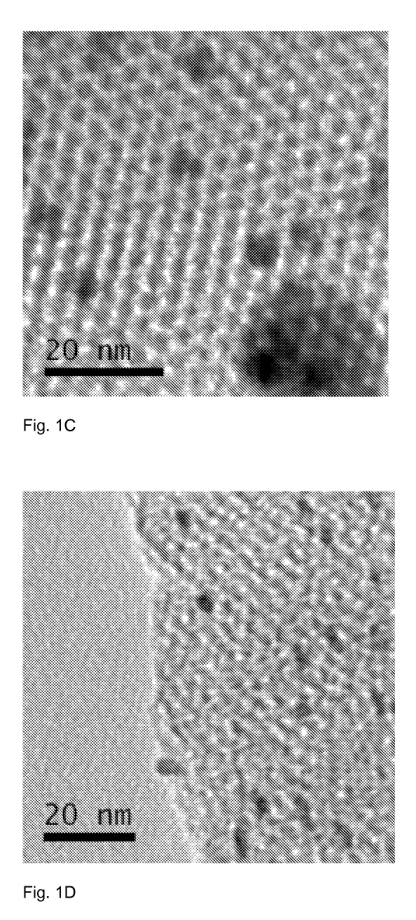

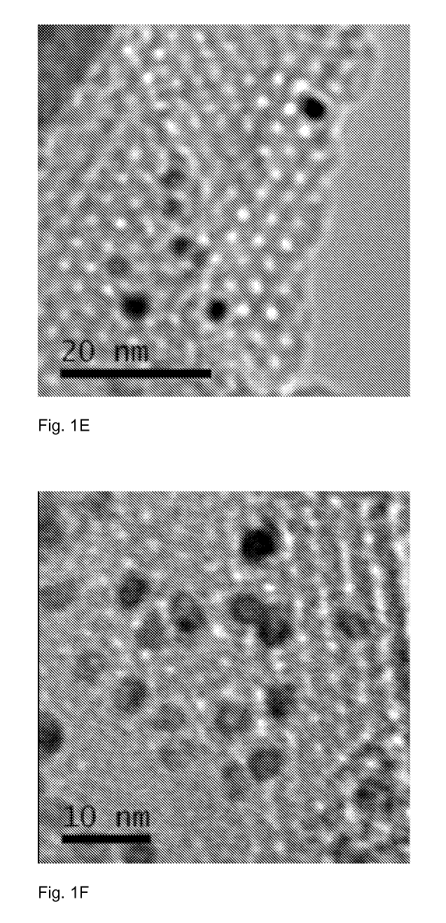

The particle sizes of the metal nanoparticles were analyzed by the transmission electron microscopy (TEM; Joel JEM-2100) in EXAMPLES 1-6. FIGS. 1A-1F are TEM images respectively illustrating the catalysts of EEXAMPLES 1-6. Statistics calculations were performed to obtain the average diameter of the nanoparticles (i.e. averaged particle size) by using an interactive imaging software (OPTIMAS5), and the results of which are summarized in Table 1. The Ru nanoparticle of EXAMPLE 1 had the smallest averaged diameter of 2.7 nm while the Rh nanoparticle of EXAMPLE 2 had the largest diameter of 7.8 nm. The average diameters of the bimetallic nanoparticles were all between the average diameters of their respective monometallic ones, which is a reasonable indication that the nanoparticles were formed as alloys or mixture of clusters.

Energy dispersive X-ray spectroscopy (EDS; Oxford 6587) was employed to provide the quantitative analyses of the nanoparticles in EXAMPLES 1-6, and the results are shown in Table 2. The overall yield in Table 2 was calculated based on the amount of the metal element(s) of the organometallic precursors. Each of the Ru and Rh monometallic nanoparticles in EXAMPLE 1 and EXAMPLE 2 exhibited a high overall yield to a level of greater than 75% while the Pd monometallic nanoparticles in EXAMPLE 3 had an overall yield of 62.4%. The bimetallic nanoparticles in EXAMPLES 4-6 exhibited overall yields between 55.9% and 60.8%. The weight percentages of the metal nanoparticles in EXAMPLES 1-3 are respectively 4.0%, 4.0% and 3.3% with respect to an overall weight of the catalysts. Even thought the organometallic precursors in EXAMPLES 4-6 were mixed in a way such that the two metal elements were of equal weight, only the ratio of Ru to Pd in EXAMPLE 5 was close to unity. In EXAMPLE 4 and EXAMPLE 6, both of the bimetallic nanoparticles exhibited a major metal element of Rh. Without being bound to any theory, it is believed that major metal in the bimetallic nanoparticles is related to the solubility of the organometallic precursors in supercritical carbon dioxide. The solubility of Rh(acac)₃ in supercritical carbon dioxide is greater than that of Ru(acac)₃ and Pd(acac)₂ according to the report of Yoda et al. (Supercrit. Fluids 44, 139 (2008)).

TABLE 2

| | | Weigh percentage | |
|---|---|---|---|
| | | of metal | Overall |
| | Catalyst | element (%) | yield (%) |
| EXAMPLE 1 | Ru/MCM-41 | Ru = 4.0 | 78.0 |
| EXAMPLE 2 | Rh/MCM-41 | Rh = 4.0 | 76.4 |
| EXAMPLE 3 | Pd/MCM-41 | Pd = 3.3 | 62.4 |
| EXAMPLE 4 | Ru-Rh/MCM-41 | Ru = 1.3; Rh = 1.8 | 59.8 |
| EXAMPLE 5 | Ru-Pd/MCM-41 | Ru = 1.5; Pd = 1.4 | 55.9 |
| EXAMPLE 6 | Rh-Pd/MCM-41 | Rh = 2.2; Pd = 0.9 | 60.8 |

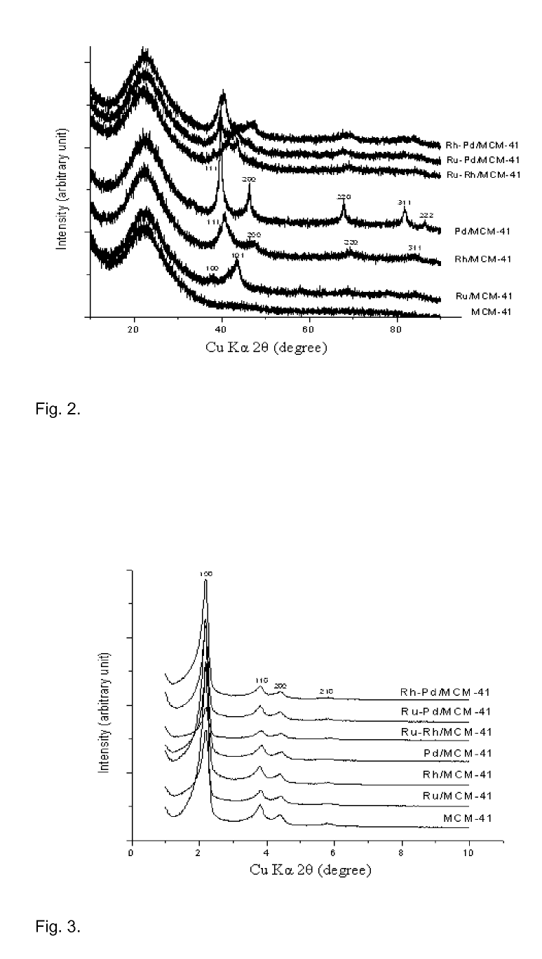

Powder and small angle X-ray diffraction (XRD; Rigaku Ultima IV) patterns are respectively shown in FIG. 2 and FIG. 3. In FIG. 2, the broad peak between 15° and 30° can be assigned to MCM-41, which is generally SiO₂. Ru has a hexagonal close-packed (hcp) structure and its major peak is (101) at 20=44.0°. On the other hand, Rh and Pd both have face-centered cubic (fcc) structures. Their major peak (111) for Rh: 20=41.1° and for Pd: 20=40.1°; secondary peak (200) for Rh: 2θ=47.8° and for Pd: 2θ=46.7°. The bimetallic Rh—Pd also showed fcc with the (111) and (200) peaks both near the center of those of Rh and Pd. This may suggest that the Rh—Pd nanoparticles have disordered alloy crystal structure. In the cases of Ru—Rh and Ru—Pd nanoparticles, the diffraction patterns showed mixtures of hcp and fcc structures. In FIG. 3, small angle diffraction patterns are used to confirm the ordered structure of MCM-41. When incorporating different metals inside the mesopores of MCM-41, the diffraction peaks all showed some kind of distortion indicating the blockage of the pore mouths by the metal nanoparticles. Moreover, the ordered structure of MCM-41 proved to be undamaged since the diffraction peaks did not show any major changes.

### Examples 7

285 mg of MCM-41 and 87 mg of Ru(cod)(tmhd)₂ were added together into a high-pressure cell for a maximum metal ratio of 5% by weight. At 150° C., 100 bar of H₂ and 100 bar of CO₂ were premixed in a gas reservoir and injected into the cell for a reaction time of 2 hours so that the organometallic precursor was reduced to ruthenium nanoparticles. After the reaction, the cell was depressurized and flushed with CO₂ for a few times to eliminate the unreacted organometallic precursors. Catalyst composed of MCM-41 and ruthenium nanoparticles was obtained, and the properties of Ru/MCM-41 catalyst were nearly identical to that in EXAMPLE 1.

### Comparative Example 1

In COMPARATIVE EXAMPLE 1, the catalyst was prepared in a manner the same as these described in EXAMPLE 1, except that no supercritical CO₂ was provided in the reaction. COMPARATIVE EXAMPLE 1 is also considered as a conventional method. The weight percentage of the Ru nanoparticles was 4.2% with respect to the overall weight of the catalyst. The averaged particle size of the Ru nanoparticles was 3.0 nm, calculated by a manner the same as EXAMPLE 1. In addition, the BET surface areas of the catalysts of COMPARATIVE EXAMPLE 1 and EXAMPLE 1 were measured, and the results are summarized in Table 3. The catalyst of COMPARATIVE EXAMPLE 1 was similar to that of EXAMPLE 1 in properties of the Ru weight percentage, particle size and BET surface area.

TABLE 3

| | Weight | | BET |
|---|---|---|---|
| | percentage | Averaged particle size of | surface area |
| | of Ru (%) | Ru nanoparticles (nm) | (m²/g) |
| EXAMPLE 1 | 4.0 | 2.7 ± 0.4 | 1068 |
| COMPARATIVE | 4.2 | 3.0 ± 0.8 | 899 |
| EXAMPLE 1 | | | |

### Catalytic Hydrogenation of p-XYLENE

### Examples 8-13

In EXAMPLES 8-13, p-xylene was hydrogenated by the catalysts prepared in EXAMPLES 1-6, respectively. 5 g of p-xylene, 45 g of methylcyclohexane (as solvent) and 50 mg of the respective catalysts were mixed in a high-pressure stainless steel cell. Hydrogen gas was then introduced into the cell at a pressure of 500 psi and a temperature of 50° C. for a reaction time of 1.67-3 hrs. The reaction products were characterized by GC/MS (HP5890II/HP5972). The results are summarized in Table 4.

TABLE 4

| | | Time | Conversion | | | TOF |
|---|---|---|---|---|---|---|
| | Catalyst | (hr) | % | Cis/Trans | TON | (hr⁻¹) |
| EXAMPLE 8 | Ru/MCM-41 | 1.67 | 100 | 3.4 | 2380 | 1428 |
| | (EXAMPLE 1) | | | | | |
| EXAMPLE 9 | Rh/MCM-41 | 3 | 27.0 | 3.0 | 628 | 209 |
| | (EXAMPLE 2) | | | | | |
| EXAMPLE 10 | Pd/MCM-41 | 3 | 0 | n/a | n/a | n/a |
| | (EXAMPLE 3) | | (no reaction) | | | |
| EXAMPLE 11 | Ru—Rh/MCM-41 | 1.67 | 29.3 | 2.5 | 882 | 529 |
| | (EXAMPLE 4) | | | | | |
| EXAMPLE 12 | Ru—Pd/MCM-41 | 3 | 41.5 | 2.5 | 1365 | 455 |
| | (EXAMPLE 5) | | | | | |
| EXAMPLE 13 | Rh—Pd/MCM-41 | 3 | 30.8 | 2.6 | 965 | 322 |
| | (EXAMPLE 6) | | | | | |
| COMPARATIVE | Ru/MCM-41 | 1.67 | 13.7 | 4.0 | 309 | 185 |
| EXAMPLE 2 | (COMPARATIVE | | | | | |
| | EXAMPLE 1) | | | | | |

In Table 4, the conversion is calculated as the number of moles of p-xylene consumed in the reaction divided by the number of moles of p-xylene originally presented. The main products produced by the hydrogenation of p-xylene comprises cis- and trans-1,4-dimethylcyclohexane. The molar ratios of cis-1,4-dimethylcyclohexane to trans-1,4-dimethylcyclohexane (i.e. “Cis/Trans” in Table 4) are shown in Table 4 as well. Furthermore, the turnover number (TON) and the turnover frequency (TOF) are addressed to compare the catalytic activities of the catalysts prepared in EXAMPLES 1-6.

EXAMPLE 8 exhibited the highest conversion of 100% in a reaction time of 1.67 hours by using the catalyst of Ru/MCM-41, whereas the conversion in EXAMPLE 10 was zero by using Pd/MCM-41 catalyst, which did not have enough activity to catalyze the hydrogenation reaction of p-xylene. The conversions in EXAMPLES 9 and EXAMPLES 11-13 were between 13.7% and 41.5%.

In EXAMPLES 8-13, the cis/trans ratios were in the region of 2.5-4.0 with the exception of EXAMPLE 10. The product favored the cis form, which is, however, a thermodynamically less favored isomer. Without being bound to any theory, it is believed that the major product of the cis form is due to the nature of the metal catalyzed hydrogenation reaction which usually undergoes the syn addition of hydrogen atoms to the unsaturated double bonds.

The Ru/MCM-41 catalysts in EXAMPLE 8 exhibited particularly high TON and TOF values among others. It was surprised that the TOF value of Ru/MCM-41 was nearly seven times higher than that of Rh/MCM-41 in EXAMPLE 9. According to the literature, the catalytic activity of different metals for the hydrogenation of benzene and alkyl-substituted benzene was reported in the order of Rh>Ru>>Pt>Pd>>Ni>Co (H. Greenfield, Ann. NY Acad. Sci. 214, 233 (1973)). A reasonable explanation would be more surface areas are available to provide active sites for the Ru/MCM-41 catalyst since the averaged particle size is about 3 times smaller than that of the Rh/MCM-41 catalyst (see Table 1). Additionally, the TOF value of Rh—Pd/MCM-41 was greater than the sum of Rh/MCM-41 and Pd/MCM-41. This suggests that a synergistic effect occurred when alloying the two metals. As shown in Table 1, the particle size of Rh—Pd nanoparticles was similar to that of Pd nanoparticles, and was significant smaller than Rh nanoparticles. In EXAMPLE 10, Pd/MCM-41 catalyst did not have enough activity to catalyze the hydrogenation reaction of p-xylene. However, the addition of Pd into Rh can reduce the particle size and possibly create more active sites of the metal surface, and thus increasing the catalytic activity. In EXAMPLES 11-12, the activity of the catalysts were less than that in EXAMPLE 8 because alloying another metal into Ru in EXAMPLES 4-5 unfavorably enlarged the particle size of the metal nanoparticles. In view of the above, it is believe that the particle size of the metal nanoparticles is an important factor in a catalytic hydrogenation reaction.

### Comparative Example 2

In COMPARATIVE EXAMPLE 2, p-xylene was hydrogenated in a manner the same as these described in EXAMPLES 8-13, except that the catalyst prepared in COMPARATIVE EXAMPLE 1 was used. The analysis results of the products produced in COMPARATIVE EXAMPLE 2 are summarized in Table 4 as well.

Unexpectedly, the TOF value in Example 8 using Ru/MCM-41 catalyst prepared in Example 1 was much greater than that in COMPARATIVE EXAMPLE 2. As mentioned hereinbefore, the Ru/MCM-41 catalyst prepared in COMPARATIVE EXAMPLE 1 was similar to that of EXAMPLE 1 in several properties such as the Ru weight percentage, particle size and BET surface area, as shown in Table 3. However, the TOF value in Example 8 was 7.7 folds of that in COMPARATIVE EXAMPLE 2. Without being bound to any theory, it is believed that the major difference of the catalyst prepared in EXAMPLE 1 from that prepared in COMPARATIVE EXAMPLE 1 is the dispersion or population density of the nanoparticles within the MCM-41 support. The Ru nanoparticles in the Ru/MCM-41 catalyst prepared in COMPARATIVE EXAMPLE 1 were observed to have some highly dense and lowly dense regions whereas the Ru/MCM-41catalyst prepared in EXAMPLE 1 exhibited a more uniform distribution of Ru nanoparticles. It is suggested that with the addition of supercritical fluid such as supercritical carbon dioxide, the nanoparticles could be redispersed into a more uniform density throughout the catalyst powder. Low dispersity could strongly retard the reaction rate since the conversion in COMPARATIVE EXAMPLE 2 was dropped to only 13.7%.

### Catalytic Hydrogenation of BPA

### Examples 14-18

In EXAMPLES 14-17, 1 g of the bisphenol A (BPA; 4,4′-isopropylidenediphenol), 50 g of solvent and 50 mg of Ru/MCM-41 catalyst prepared in Example 7 were all added into a high-pressure autoclave. The solvents in EXAMPLES 14-17 were respectively cyclohexane, ethylacetate, isopropanol and water. Hydrogen gas of 50 bars was then introduced into the autoclave at a temperature of 75° C. for a reaction time of 5 hours. The experimental apparatus used herein was a semi-batch system. The products were analyzed by gas chromatography-mass spectrometry (GC-MS; HP589011/HP5972), and the results are summarized in Table 5. The turnover frequency (TOF) in Table 5 is calculated as the number of moles of hydrogen gas consumed in one hour (1 mole of H₂ for 1 mole of C═C double bond) divided by the number of moles of the metal in the catalyst (also refers to the term “turnover frequency associated with hydrogen” described hereinbefore).

TABLE 5

| | | H₂ | H₂ | Time | Conversion | HBPA yield | TOF |
|---|---|---|---|---|---|---|---|
| | Solvent | P (bar) | T (° C.) | (hr) | % | (%) | (hr⁻¹) |
| EXAMPLE 14 | cyclohexane | 50 | 75 | 5 | 21.3 | 7.9 | 41 |
| EXAMPLE 15 | ethylacetate | 50 | 75 | 5 | 12.7 | 0.6 | 19 |
| EXAMPLE 16 | isopropanol | 50 | 75 | 5 | 44.4 | 8.7 | 74 |
| EXAMPLE 17 | water | 50 | 75 | 5 | 97.0 | 51.0 | 207 |
| EXAMPLE 18 | water | 50 | 85 | 4 | 99.6 | 91.9 | 342 |

In EXAMPLES 14-16, the conversion of BPA was merely between 12.7% and 44.4%, and the product, 4,4-isopropylidenedicyclohexanol (HBPA), had a yield of 0.6% to 8.7%. The turnover frequency (TOF) was between 19 hr⁻¹ and 74 hr⁻¹.

It is unexpected that the reaction proceeded most rapidly in water in connection with EXAMPLE 17, in which BPA conversion, HBPA yield and TOF were respectively 97.0%, 51.0% and 207 hr⁻¹. The reactant BPA is almost insoluble in water and cyclohexane, whereas it is completely soluble in ethyl acetate and isopropanol. Without being bound to any theory, it is believed that water provides a suitable environment for the silica-based catalyst to disperse thoroughly in the reaction medium because the hydroxyl groups on the silica surface could form hydrogen bonding with the water molecules. The hydrogen bonds could help the water molecules surround the catalyst surface, and thus creating a better dispersion of the catalyst.

In EXAMPLES 18, BPA was hydrogenated by the same method as described in EXAMPLE 17, except that the reaction was performed at a temperature of 85° C. for a reaction time of 4 hours. The BPA conversion was increased to 99.6%, and the HBPA yield and TOF were respectively 91.9% and 342 hr⁻¹.

### Hydrogenation of BPF and Benzoic Acid in Water

### Examples 19

In this example, bisphenol F (BPF; 4,4′-methylenediphenol) was hydrogenated in a manner the same as these described in EXAMPLE 18. Briefly, 1 g of the bisphenol F, 50 g of water and 50 mg of Ru/MCM-41 catalyst prepared in Example 7 were added into a high-pressure autoclave. Hydrogen gas of 50 bars was then introduced into the autoclave at a temperature of 85° C. for a reaction time of 4 hours. The BPF conversion and the TOF (refers to “turnover frequency associated with hydrogen”) were respectively 94.2% and 337 hr⁻¹ in this example.

### Examples 20

In this example, 1 g of benzoic acid, 50 g of water and 50 mg of Ru/MCM-41 catalyst prepared in Example 7 were added into a high-pressure autoclave. Hydrogen gas of 50 bars was then introduced into the autoclave at a temperature of 85° C. for a reaction time of 1 hours. The conversion of benzoic acid and the TOF (refers to the term “turnover frequency associated with hydrogen” described hereinbefore) were respectively 100% and 1329 hr⁻¹ in this example.

### Durability of Catalyst

### Example 21

In this example, the durability or recyclability of the Ru/MCM-41 catalyst prepared in EXAMPLE 7 was evaluated. The Ru/MCM-41 catalyst was used to hydrogenate BPA by the same method as described in EXAMPLE 18, except that the hydrogenation reaction was performed for 3 hours. After the hydrogenation reaction, the catalyst was recycled by the method provided by T. Witula et al. (T. Witula, K. Holmberg, Langmuir 21 (2005) 3782). Briefly, water in the resulting slurry was removed by using a rotary evaporator. Afterwards, ethanol was added to the slurry type residue to dissolve the products so that the catalyst could be separated from ethanol solution by filtration. The recycled catalyst was used to hydrogenate BPA in another run. Three runs of the hydrogenation reaction of the BPA were repeated in this example.

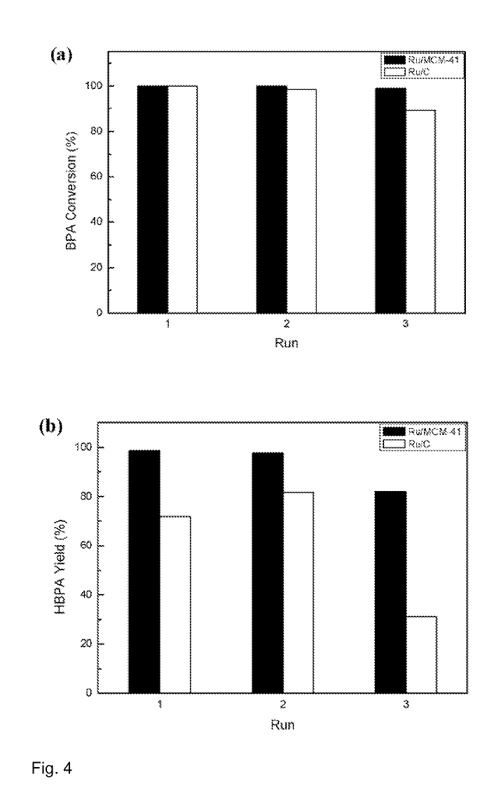

FIG. 4a and FIG. 4b respectively illustrate the BPA conversion and the HBPA yield for the three runs of the hydrogenation reaction. The Ru/MCM-41 catalyst prepared in EXAMPLE 7 exhibited an excellent BPA conversion of about 100% in all of the three runs. The HBPA yield slightly decreased to about 80% in the third run.

### Comparative Example 3

In this comparative example, the durability of a commercial catalyst of ruthenium on activated carbon (5% Ru/C, Strem) was evaluated by the same method as described in EXAMPLE 21. The BPA conversion and the HBPA yield are illustrated in FIG. 4a and FIG. 4b as well. The BPA conversion associated with Ru/C catalyst decreased to about 90% in the third run. The Ru/C catalyst exhibited a HBPA yield of about 70-80% in the first and second runs. However, the HBPA yield dropped to about 30% in the third runs.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A method for manufacturing a catalyst having a plurality of metal nanoparticles dispersed therein, comprising:
allowing an organometallic precursor to be in contact with a mesoporous support, wherein the organometallic precursor comprises at least one material selected from the group consisting of ruthenium-containing compound, rhodium-containing compound and palladium-containing compound; and
reducing the organometallic precursor in the presence of a supercritical fluid with a reductant, so that the organometallic precursor is reduced to the metal nanoparticles.

2. The method of claim 1, wherein the organometallic precursor is a material selected from the group consisting of ruthenium acetylacetonate [Ru(acac)₃], rhodium acetylacetonate [Rh(acac)₃], palladium acetylacetonate [Pd(acac)₂] and any combination thereof.

3. The method of claim 1, wherein the organometallic precursor is a material selected from the group consisting of bis(2,2,6,6-tetramethyl-3,5-heptanedionato)(1,5-cycloctadiene)ruthenium [Ru (cod)(tmhd)₂], acetylacetonato(1,5-cyclooctadiene) rhodium [Rh(cod)(acac)] and palladium hexafluoroacetylacetonate [Pd(hfac)₂] and any combination thereof.

4. The method of claim 1, wherein a molar ratio of the reductant to the supercritical fluid is about 0.1:1 to 1:1.

5. The method of claim 1, wherein a molar ratio of the reductant to the organometallic precursor is greater than or equal to about 10:1.

6. The method of claim 1, wherein the metal nanoparticles exists in a concentration of 0.5-10% by weight of the catalyst.

7. The method of claim 1, wherein the mesoporous support is made of silica, and comprises a hexagonal array of mesopores.

8. The method of claim 1, wherein each of the metal nanoparticles has a diameter of less than about 10 nm.

9. The method of claim 1, wherein the supercritical fluid is supercritical carbon dioxide, and the reductant is hydrogen.

10. The method of claim 1, wherein the step of reducing the organometallic precursor to the metal nanoparticles is performed at a temperature of about 100° C. to about 300° C.

11. The method of claim 1, wherein the step of reducing the organometallic precursor to the metal nanoparticles comprises exposing the organometallic precursor and the mesoporous support to a mixture of the supercritical fluid and the reductant.

12. The method of claim 1, wherein the step of reducing the organometallic precursor to the metal nanoparticles comprises the following steps in sequence:
subjecting the organometallic precursor and the mesoporous support in a container;
introducing the supercritical fluid into the container; and
introducing the reductant into the container having the supercritical fluid.

13. The method of claim 1, wherein the step of allowing the organometallic precursor in contact with the mesoporous support comprises:
mixing the organometallic precursor and the mesoporous support with a solvent at atmosphere, so that the organometallic precursor is dissolved in the solvent; and
removing the solvent such that the organometallic precursor is adsorbed on the mesoporous support.

14. A catalyst produced according to the method of claim 1.

15. A method for hydrogenating an aromatic compound, comprising:
providing a catalyst of claim 14;
mixing the aromatic compound and the catalyst with a solvent and hydrogen, and thus allowing the aromatic compound to be hydrogenated by the hydrogen in the presence of the catalyst and the solvent.

16. The method of claim 15, wherein the solvent is water.

17. The method of claim 16, wherein the step of mixing the aromatic compound and the catalyst with the water and the hydrogen comprising:
allowing the aromatic compound and the catalyst to be in contact with the water, and thus forming a mixture; and
introducing the hydrogen into the mixture.

18. The method of claim 16, wherein the organometallic precursor comprises a material selected from the group consisting of ruthenium acetylacetonate [Ru(acac)₃], rhodium acetylacetonate [Rh(acac)₃] and a combination thereof.

19. The method of claim 16, wherein the organometallic precursor comprises a material selected from the group consisting of bis(2,2,6,6-tetramethyl-3,5-heptanedionato)(1,5-cycloctadiene)ruthenium [Ru (cod)(tmhd)₂], acetylacetonato(1,5-cyclooctadiene) rhodium [Rh(cod)(acac)] and a combination thereof.

20. The method of claim 16, wherein the mesoporous support is made of silica, and the aromatic compound is a material selected from the group consisting of p-xylene, 4,4-isopropylidenediphenol, 4,4-methylenediphenol and benzoic acid.
